(19) United States
(12) Patent Application Publication
(10) Pub. No.: US 2023/0172989 A1
(43) Pub. Date: Jun. 8, 2023

(54) SYSTEM AND A METHOD FOR OBTAINING AN IMPROVED PLASMA EXTRACT

(71) Applicant: ESTAR TECHNOLOGIES LTD, Holon (IL)

(72) Inventor: Aaron ESTERON, Holon (IL)

(73) Assignee: ESTAR TECHNOLOGIES LTD, Holon (IL)

(21) Appl. No.: 17/916,328

(22) PCT Filed: Mar. 30, 2021
(86) PCT No.: PCT/IL2021/050359

§ 371 Date: Sep. 30, 2022

Related U.S. Application Data
(60) Provisional application No. 63/001,676, filed on Mar. 30, 2020.

(51) Int. Cl. A61K 35/19; C12N 5/0786; C12N 5/078

(52) U.S. Cl. CPC A61K 35/19; C12N 5/0645; C12N 5/0644; C12N 2500/05

(57) ABSTRACT

A system and a method are disclosed for obtaining a plasma composition enriched with bioactive molecules such as growth factors and cytokines and depleted in contaminants such as viruses.

### RELATED APPLICATION DATA

This application claims priority from U.S. Provisional Patent Application No. 63/001,676, filed on Mar. 30, 2020, which is incorporated herein by reference in its entirety.

### BACKGROUND OF THE INVENTION

U.S. Pat. Nos. 9,962,480 and 10,617,812, by the inventor herein, include embodiments directed to systems and methods for obtaining cellular samples enriched with cells, such as a platelet rich plasma (PRP), including separations using centrifugation of a blood sample in a centrifugation tube using a density separation medium. The disclosure of these patents is incorporated by reference.

U.S. Pat. Nos. 10,167,310 and 10,519,196, also by the inventor herein, include embodiments directed to systems and methods for obtaining a plasma fraction enhanced in Interleukin Receptor Antagonist (IL-1Ra). In embodiments, the disclosed methods involve subjecting a blood sample in a blood collection tube to centrifugation and incubating the plasma fraction to enrich the fraction in IL-1Ra.

However, plasma compositions enhanced in specific anti-inflammatory cytokines and growth factors derived from PRP are not generally available. Recombinant cytokines, including IL-1Ra, are commercially available However, such compositions differ substantially from a composition obtained from PRP or other blood plasma fraction.

### SUMMARY OF THE INVENTION

The present invention represents an advance on the known prior art in terms of developing and perfecting the manipulation and extraction of cellular samples; increasing the content of bioactive molecules in such cellular samples; reducing the contaminants in such samples, including viral contaminants; increasing the types of cellular samples that may be obtained; and widening the therapeutic applications for such compositions.

In one aspect, the invention relates to a system and a method for obtaining a plasma composition enriched with bioactive molecules such as growth factors and cytokines which are derived from a defined cellular fraction, wherein the plasma composition may be depleted in contaminants, such as viruses and their derivatives, and to methods of making and methods of use.

In one aspect, the invention is embodied as a system for obtaining blood plasma enriched in bioactive molecules, the system comprising a cell suspension collection tube containing a gel separation medium and incubation beads, said collection tube adapted to be centrifuged to separate a blood sample and adapted to be incubated for 30 minutes to 24 hours.

In one aspect, the invention is embodied in a method for obtaining blood plasma enriched in bioactive molecules, comprising: providing a cell suspension collection tube containing a gel separator and incubation beads; providing a blood sample or cellular suspension in the cell suspension collection tube; centrifuging the cell suspension collection tube to obtain three fractions in the tube, including: a first fraction containing cells and beads and adapted to be discarded; a second fraction comprising the gel; and a third fraction containing cells and adapted to be recovered; and extracting a cellular fraction containing blood plasma from the third fraction, wherein said extracting includes increasing a concentration of bioactive molecules in the cellular fraction.

In embodiments, a composition according to the invention may be obtained from PRP or other blood plasma fraction by centrifugation and may be enriched in a plurality of anti-inflammatory cytokines and anti-inflammatory growth factors selected from the group consisting of alpha-2-macroglobulin (A2M), hepatocyte growth factor HGF, transforming growth factor beta TGFβ, platelet derived growth factor PDGF, Interleukin 10 IL₋₁₀, Interleukin 4 IL₋₄, Interleukin receptor antagonist IL-1Ra, Interleukin 4 receptor antagonist IL₄Ra, Interleukin 11 IL₋₁₁, and Interleukin 13 IL₁₃ by activating monocytes retained in a plasma fraction following centrifugation.

In one aspect, the invention is embodied as a method for obtaining blood plasma enriched in bioactive molecules. The method comprises providing a blood sample to a cell suspension collection tube containing a gel separator and activators for induction of bioactive molecules; incubating the collected blood sample in the collection tube for 30 minutes to 24 hours; centrifuging the cell suspension collection tube to obtain three fractions in the tube, including: a first fraction containing cells and adapted to be discarded; a second fraction comprising the gel; and a third fraction containing cells and enriched in bioactive molecules, and adapted to be recovered; and collecting the fraction enriched in bioactive molecules.

In embodiments, activating a defined fraction of monocytes in a plasma fraction obtained from blood separation, for example by contacting a sample containing monocytes with glass beads and/or coated or uncoated polymer beads, releases growth factors and cytokines. In embodiments, the cell suspension collection tube may be incubated before and/or after centrifugation to increase a concentration of desired bioactive molecules in the fraction.

The collected enriched bioactive fraction may be further concentrated through filtration with water adsorbent materials (hemoconcentration).

The collected enriched bioactive fraction may be prepared for administration by injection or inhalation, wherein such administration can be delivered at the point of care or up to six months from cell collection. In embodiments, a composition is delivered directly to a damaged lung tissue through inhalation or spraying.

In embodiments, the fraction enriched in bioactive molecules may be reduced in contaminants, including viruses, by applying specified physical, mechanical, physical-mechanical, and/or chemical stress to a separated sample.

### BRIEF DESCRIPTION OF THE FIGURES

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 depicts a cell suspension tube used in embodiments of the invention;

FIG. 2 schematically depicts a centrifuge adapted to receive at least one collection tube;

FIG. 3 schematically depicts gentle resuspension of a collection tube having fractions separated by centrifugation;

FIG. 4 depicts withdrawal of a cellular fraction obtained from a separated sample;

FIG. 5 depicts a collection tube provided with incubation beads according to another embodiment of the invention;

FIG. 6 schematically depicts incubating the collection tube of FIG. 5 at 37° C. according to an embodiment of the invention;

FIG. 7 schematically depicts centrifugation of a collection tube provided with incubation beads and blood sample;

FIG. 8 schematically depicts withdrawal of a cellular fraction obtained from a centrifuged sample of FIG. 7;

FIG. 9 depicts a stoppered collection tube comprising incubation beads and separation gel, to which blood or a cellular suspension is provided according to another embodiment of the invention;

FIG. 10 depicts incubation of the collection tube depicted in FIG. 9;

FIG. 11 depicts centrifugation of the collection tube in FIG. 10;

FIG. 12 schematically depicts gentle resuspension of a collection tube having fractions separated by centrifugation and enhanced in bioactive molecules;

FIG. 13 depicts withdrawal of a cellular fraction from collection tube;

FIG. 14 and FIG. 15 are flowcharts depicting generalized procedures for carrying out embodiments of the invention; and

FIG. 16 shows levels of white blood cells obtain in separated blood plasma after different periods of incubation according to the invention.

### DETAILED DESCRIPTION OF THE INVENTION

The system according to embodiments of the invention may be closeable to the atmosphere and includes a cell suspension collection tube adapted to receive a blood sample or other cell suspension. The collection tube is adapted to be centrifuged and may comprise a separator medium, such as an inert polymer gel of defined composition and density. The tube may further be provided with anticoagulant in an amount sufficient to prevent coagulation of blood in the blood sample or other cell suspension. In embodiments, the anticoagulant may be omitted.

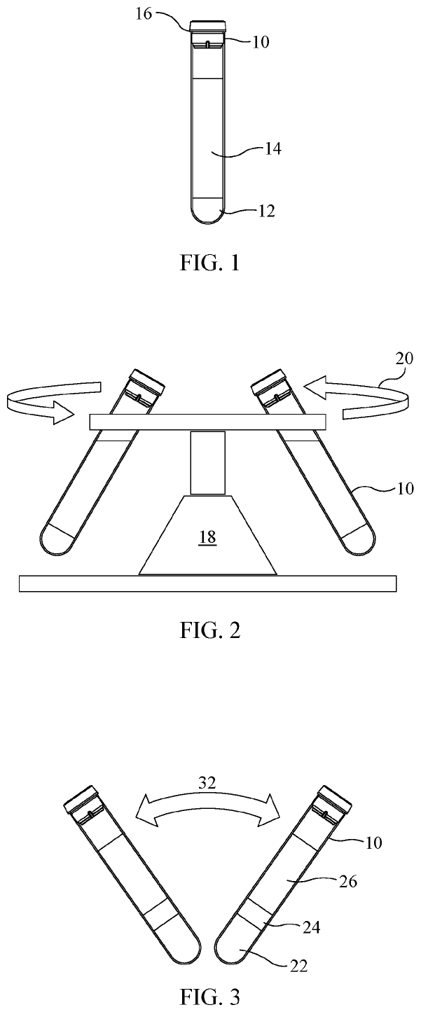

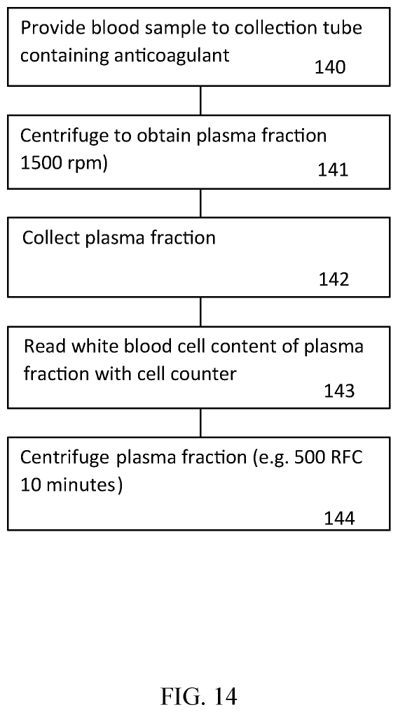

FIG. 1 depicts a cell suspension tube 10 provided with a stopper 16 and a separation medium 12. In embodiments the tube is a vacuum tube or otherwise may be adapted to receive a predetermined quantity of blood 14 (or cellular suspension) and optionally may be provided with an anticoagulant (FIG. 14, step 140). FIG. 2 schematically depicts a typical centrifuge 18 adapted to receive the collection tubes and supply sufficient RPMs 20 to impart a force of, for example, 500 g-5000 g (FIG. 14, step 141).

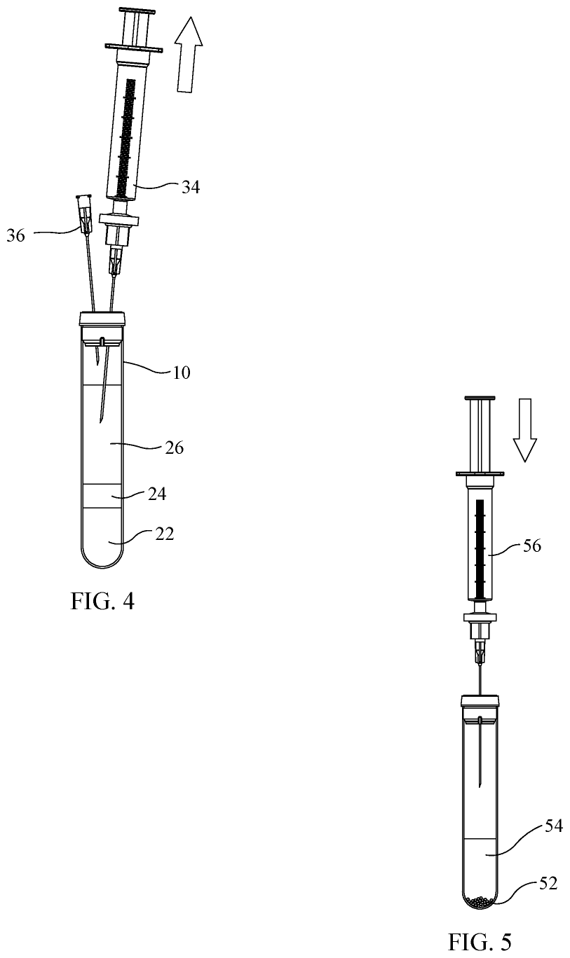

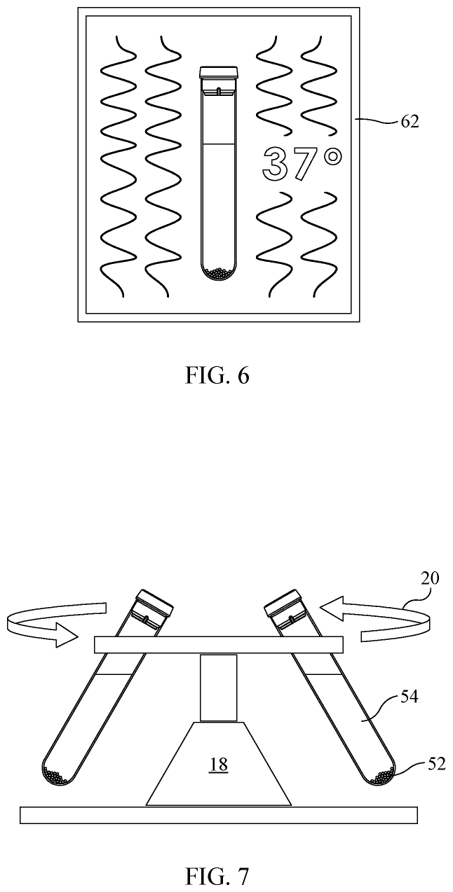

The system is designed such that following centrifugation of blood, three fractions are obtained as shown in FIG. 3: a first (discarded) cellular fraction 22 containing red blood cells, granulocytes, neutrophils and typically a portion of monocytes; a second fraction 24 containing gel; and a third cellular fraction 26 from which desired bioactive molecules are extracted after a gentle resuspension 32 (FIG. 14, step 142). Depending on the embodiment, the white blood cell count may be determined at this stage with a cell counter (FIG. 14, step 143), and/or the third cellular fraction may be further concentrated, for example in a hemoconcentrator which removes water, a further centrifugation (FIG. 14, step 144), or may be used without further concentration. The third cellular fraction may be optionally subjected to cell culturing for enrichment in defined cells, such as monocytes and lymphocytes. FIG. 4 depicts extracting a cellular fraction containing blood plasma from the third fraction 26 with syringe 34 utilizing a venting needle 36 comprising a filter, according to an embodiment of the invention.

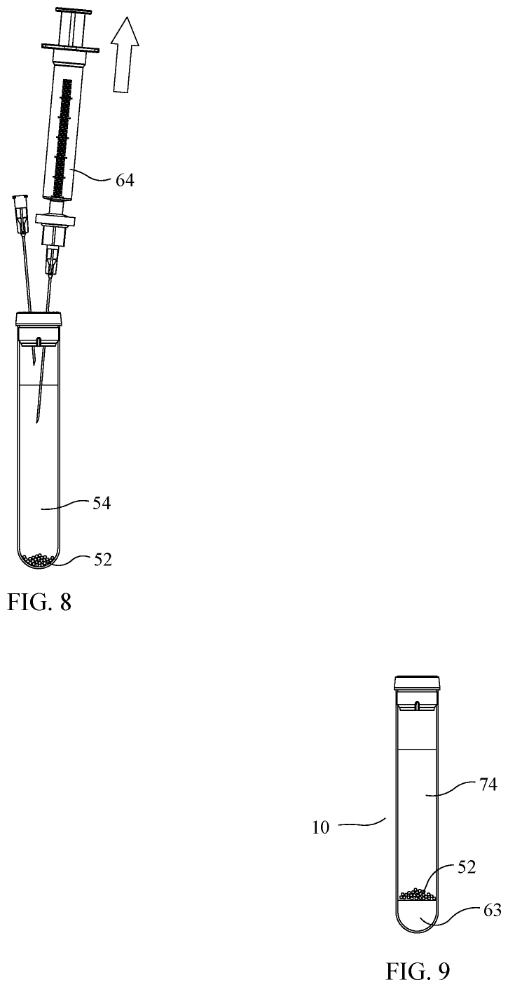

In embodiments, a collection tube may be provided with glass beads and/or coated or uncoated polymer beads adapted to incubate cells in the blood plasma, before or after centrifugation. In FIG. 5, a blood sample 54 is supplied to a collection tube with syringe 56. The collection tube is pre-supplied with incubation beads 52, for example glass beads and/or coated or uncoated polymer beads. The collection tube may be placed in incubator 62 with the beads and the blood sample under the conditions shown in FIG. 6. Following centrifugal separation shown in FIG. 7, extraction may be performed with extraction syringe 64 as shown in FIG. 8.

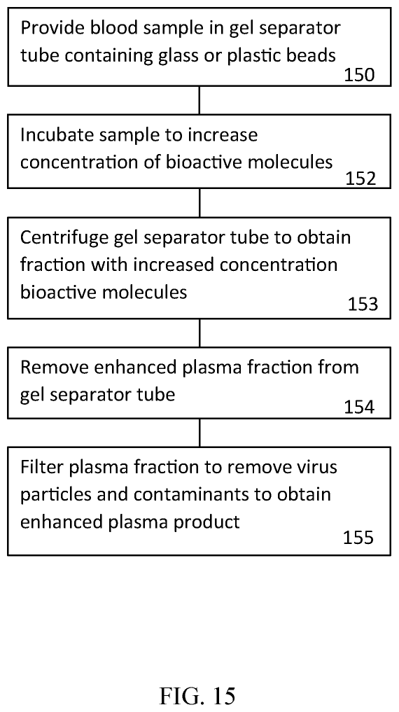

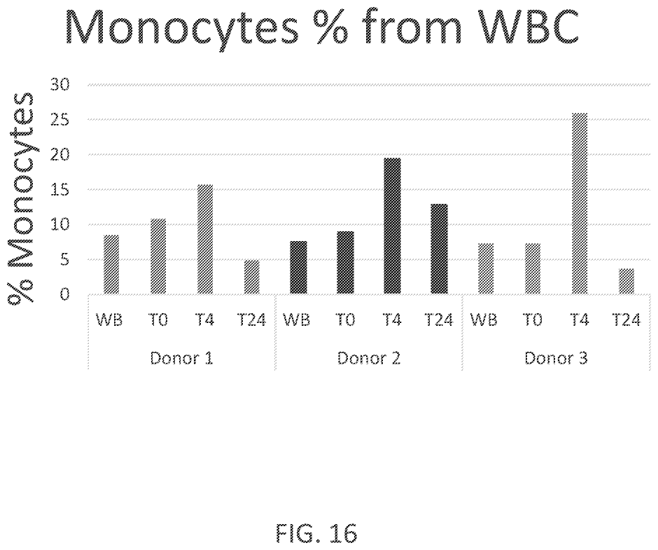

In one embodiment of the invention, a system for obtaining a blood plasma fraction enriched in bioactive molecules comprises a cell suspension collection tube containing a gel separation medium and incubation beads. FIG. 15 depicts a sequence wherein incubation is performed in a gel separator tube containing beads. FIG. 9 depicts collection tube 10 provided with separator gel 63 and a quantity of beads 52 (FIG. 15, step 150), which may be for example glass beads (for example, borosilicate glass beads), and/or coated or uncoated polymer beads (for example, acrylamide beads).

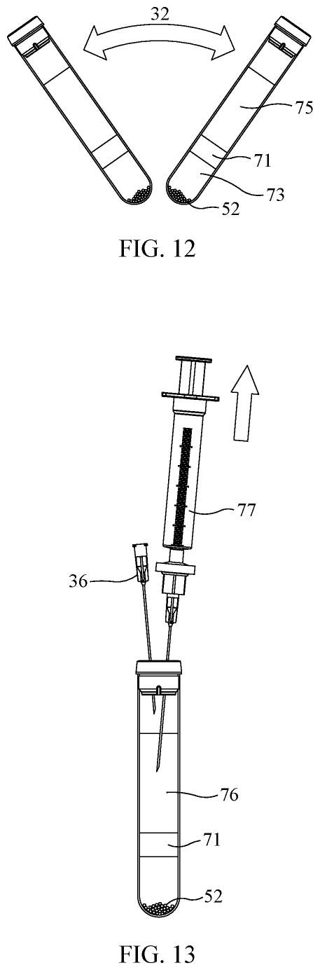

As shown in FIG. 12, the density of gel separation medium 71 is selected to settle below a predefined fraction of monocytes (e.g., white blood cells) retained in a sample 75 separated by centrifugation (FIG. 15, step 153), so that the monocytes may be activated to release desired anti-inflammatory biomolecules. After separation, beads 52 are captured in fraction 73 with red blood cells and granulocytes. Separation medium 71 may be of any kind known per se, for example gel polymers. based on polyacrylic, polyolefin or polyester with a density in the range of about 1.04 to 1.09 g/cm³, which may further include silica or other fillers. A gel separation medium used to obtain PRP in a centrifugal separation might have a density be a range of 1.04 to 1.06 g/m³ to retain a minimal amount of white blood cells in a desired PRP fraction. However, to obtain sufficient monocytes in a plasma fraction separated by centrifugation to enhance release of anti-inflammatory cytokines, the density of the separator gel may be in a range of 1.07 to 1.09 g/cm³.

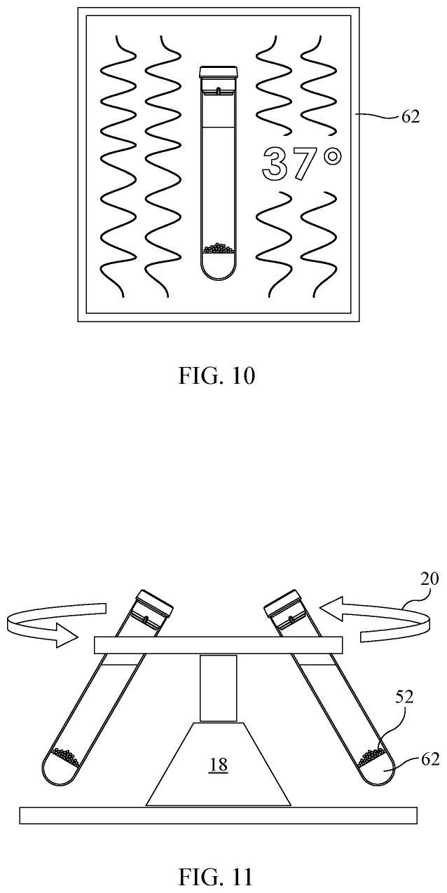

In a method according to embodiments of the invention, a cell suspension collection tube containing a gel separator and incubation beads described above is provided with a blood sample 74. The sample may be incubated in the presence of the beads as shown in FIG. 10 (FIG. 15, step 152). For the example shown, incubation was conducted at body temperature (37° C.), though incubation may be conducted in a temperature range from room temperature to 50° C., to 60° C., or higher.

Incubation in the presence of beads activates the monocytes to release cytokines. The combination of cytokines produced may vary according to the defined cells retained in the faction and the incubation. In the embodiment shown in FIG. 11, the incubated sample is centrifuged to allow beads 52 to settle into a first fraction 73 containing cells (e.g., red blood cells, granulocytes) and adapted to be discarded. A second fraction 71 comprising the gel sits between the first fraction and a third fraction 75 containing cells and adapted to be recovered, for example with syringe 77 (FIG. 13, FIG. 15, step 154).

Incubation may proceed for a time sufficient to maximize monocyte concentration in the third fraction, for example from 30 minutes to 24 hours, in embodiments 2 to 24 hours, and in embodiments 6 to 9 hours.

The third cellular fraction may be manipulated such that the cellular fraction is enhanced using defined chemical-biological or physical-mechanical modalities to induce the release of bioactive molecules such as growth factors and cytokines, or to reduce a concentration of contaminants (FIG. 15, step 155).

Cytokines that may be obtained and concentrated from blood plasma in the third cellular fraction include, without limitation, Interleukin receptor antagonist (IL-1Ra), Interleukin 10 (IL₋₁₀), Interleukin 4 (IL₋₄), Interleukin 4 receptor antagonist (IL₋₄Ra), Interleukin 11 (IL₋₁₁), Interleukin 13 (IL₁₃), transforming growth factor TGFβ, and A2M which are anti-inflammatory.

Interleukin-1 (IL-1) is produced from multiple cell types including activated monocytes. This cytokine is involved in inflammatory and immune responses in the body, including many chronic inflammatory conditions. The activity of IL-1 can be inhibited by binding to the IL-1 receptor with an interleukin-1 receptor antagonist (IL-1Ra). IL-1Ra can be produced from monocytes and polymorphonuclear cells (PMNs).

Alpha-2-Macroglobulin (A2M) is a large (720 KDa) plasma protein found in the blood. It is mainly produced by the liver, and also locally synthesized by macrophages, fibroblasts, and adrenocortical cells. In humans, it is encoded by the A2M gene.

The growth factors that are considered anti-inflammatory and may be enhanced in the third fraction are hepatocyte growth factor HGF, TGFβ and platelet derived growth factor PDGF (which inhibits the release of pro-inflammatory cytokines). These anti-inflammatory species may be present in combination in blood plasma which may be enhanced by incubation of desired cells before or after a gel separation of a blood sample.

Growth factors may be considered anti-inflammatory where they are inherently anti-inflammatory or where they inhibit proinflammatory cytokines, such as insulin growth factor (IGF). Enhancing the activity or concentration of the desired cytokines in a plasma product derived from PRP has the advantage that a plurality of such species is present in a composition which is not the case with a composition based on a single recombinant protein.

A fraction containing plasma that has been obtained by centrifugal separation in a separation tube is preferably reduced in some contaminants, including some inflammation-causing species. In embodiments, a concentration of anti-inflammatory proteins present in the separated plasma may be concentrated by excluding an amount of water from the plasma in a hemoconcentrator.

The third cellular fraction may be manipulated to eliminate contaminants such as viruses and virus derivatives using defined methodologies, including inactivation of undesired components, particularly viral components, by mechanical-physical stress, by filtration, and chemically.

The elimination of viral particles can be achieved by using nanofiltration configured to eliminate the viral particles and not the proteins. The nano filters can range from a pore size of about 15 nm up to about 35 nm, which is capable of eliminating, for example, Covid-19 virus. Removing virus particles enables the use of non-autologous plasma in cases where autologous plasma is not sufficient—Covid-19 has diameter of approximately 100 nm so a filter must be capable of eliminating such particles while allowing passage of the proteins of interest, Epstein-barr viruses (EBV) are also common, having diameters in a range of 80 nm to 120 nm and a filter may be sized accordingly.

The virus-eliminating filter can be composed of cellulose or polyvinylidene fluoride (PVDF) that enables transmission through the filter of proteins ranging from 15 kDa to 720 kDa.

In embodiments of the invention, blood may be collected in the collection tube directly from a donor or from a non-autologous source using routine procedures well known in the art of phlebotomy. Alternatively, other cellular suspensions, such as blood fractions, may be introduced into the collection tube using routine methods.

Examples of suitable collection tubes that may be used in the present invention include the Tropocells® and Cellenis® tubes produced by Estar Technologies Ltd. (Holon, Israel) and which may be centrifuged at the recommended conditions for these tubes to provide the desired separation into three layers. In embodiments, collection tubes according to the invention are made of glass, treated with silicone emulsion and/or Teflon emulsion on an inner surface thereof. In embodiments, plastic suspension collection tubes, comprising for example Polyethylene Terephthalate (PET), polyethylene terephthalate glycol (PETG), polyamide (PA) or modified polyamide (MPA) may be used, likewise treated with silicone emulsion and/or Teflon® (Polytetrafluoroethylene) emulsion coated on an inner surface thereof. In some embodiments, the test tube has a layered structure such that the interior wall of the test tube comprises a polypropylene or polyethylene tubes with or without a closed end in a section of the tube that may serve as an inner hydrophobic liner.

Collection tubes can further be provided with a special stopper. By way of a non-limiting example the cover may be made of butyl rubber or its halo derivative formulations at hardness between 40-60 Shore A. The hardness assures stable vacuum for at least the shelf life of the collection tube which can be between 18-24 months. The tube can be vacuum or non-vacuum. For non-vacuum tubes, blood is drawn by standard means, such as a needle and syringe and transferred into the tube through a security opening, which sustains a closed system environment. In embodiments, the stopper may be adapted for insertion of a vented needle having a filter to assist in extractions from the tube.

The tube may be provided with an anticoagulant layer, such as buffered citrate, ACD, modified ACD (citric acid/citrate dextrose), ACD-A, MNC7, MACD7, heparinate salts, EDTA salts, iodo acetate salts, oxalate salts, fluoride salts as water solutions or lyophilized material or wet or dry spray on an inner wall of the collection tube. Any type of anticoagulant designed for preventing clotting of blood may be used, and in some embodiments may be omitted.

As discussed below, activator compositions may be provided in the tube when blood is drawn, combined later, or in embodiments omitted.

The cellular suspension or blood may be autologous or non-autologous for the intended recipient. A non-autologous blood is obtained by a blood donation and further manipulated as described for an autologous blood cell suspension.

It is another object of the invention to provide a method wherein the third fraction or layer can be comprised of monocytes and lymphocytes and is optionally further enriched in these cellular components by cell culturing, and purified and manipulated thereafter.

An autologous cellular platelets fraction, can be obtained from a tube containing anticoagulant in a defined concentration resulting in a plasma layer, or a tube that does not contain anticoagulant which results in a solid gel-like activated platelet derived rich fibrin matrix.

The present invention also provides a method for obtaining a high concentration of bioactive molecules such as growth factors and cytokines that are released from the enriched third phase cellular fraction by employing different methodologies that are employed separately or in combination.

For example, the third cellular fraction may be further manipulated after centrifugation to release the growth factors and cytokines from platelets that are entrapped within their granules or within the matrix. The release of the growth factors can be achieved by a chemo-biological or mechano-physical stimulation.

For chemo-biological stimulation the following activators may be employed: adenosine diphosphate (ADP), CaCl2), exogenous thrombin, autologous thrombin and matrix compounds such as collagen and hyaluronic acid that may be derived from either vertebrates or non-vertebrates, or produced synthetically or in recombinant procedures.

Mechano-physical stimulation relates to a high pressure or applying a different osmotic pressure solution, extreme temperature change or sonication. Bioactive molecules such as growth factors and cytokines can be obtained by freezing-thawing cycles, or any procedure that produces a structural membrane fracture that eventually leads to the release of the bioactive molecules.

A third cellular fraction obtained from a non-anticoagulant-containing collection tube is referred to as platelet rich fibrin (PRF) and is considered as an activated form of the platelet rich plasma concentrate. The release of growth factors from the activated form of PRP can be performed by a short or prolonged incubation in a defined temperature such 37° C. or 56° C. for a time duration of 15 to 60 minutes.

It is another object of the present invention to provide a system and method wherein the serum or plasma enriched with purified bioactive molecules that is obtained can be used at the point of care or maintained in a preservative temperature such as −80° C. for a defined period such as one month for repeatable treatment.

It is another object of the present invention to provide a method for preservation of bioactive derived cell suspension molecules wherein said biomolecule derived cell suspension can also be lyophilized in a defined condition with a defined humidity which enables also the pathogen depletion or pathogen inactivation.

The present invention further provides a method for virus depletion in the enriched bioactive molecules fraction obtained from the third cellular fraction in order to avoid possible contamination of regenerated tissue by contaminated plasma.

A method for virus depletion refers to either a mechano-physical stress, filtration, or chemical stress obtained by a defined chemical solutions that comprises solvents, surfactants, detergents, or a combination of thereof.

A mechanico-physical stress can be achieved by employing a defined low pressure that ranges from 10-12 bars combined with a defined high temperature that ranges between 60° C.-75° C. Pathogen inactivation procedures that selectively maintain the desired proteins' properties and content within a plasma or blood products can be also achieved by ultrashort pulsed laser irradiation or lyophilization in a defined humidity conditions.

In embodiments, the third cellular fraction enriched in bioactive molecules is passed through a defined pore sized matrix column that entraps contaminants within the column and enables the passage of desired bioactive molecules that were enriched in the previous step.

Chemical stress may be a result of solvent or surfactant solution that destroys the nature and characteristics of the contaminant agent structure, which can be used separately or in combination, including for example, tri-n-butyl phosphate, Triton X-100, and Tween 80. In embodiments, a volume concentration of these agents ranges from 0.5% to 1%. non-physiological pH solution adjusted to inactivate the virus infectivity while maintaining the effectivity of the plasma biomolecules content.

In embodiments, the third cellular fraction containing biologically active molecules may be adapted and used to treat and control inflammation conditions and tissue destruction related to chronic or prolonged tissue damage affecting soft and hard tissue destruction, including but not limited to COPD, ulcerations, bone fractures and cartilage inflammation.

In some embodiments, the invention provides a system and method for providing a composition that may be used for treating or aiding in the treatment of a subject with a respiratory condition or disorder, including viral conditions, or where a viral infection such as Covid-19 or other SARS-COV viruses, has caused, affected or worsened a respiratory condition or disorder. The method may include the steps of obtaining blood; separating the blood to obtain a platelet-containing fraction, optionally having an increased concentration of monocytes, derived anti-inflammatory proteins and/or lymphocyte-derived cytokines, activating the platelet-containing fraction to enrich the fraction in growth factors and other bioactive molecules, and depleting or removing viruses and viral components from the fraction to form a growth factor-enriched and virus-depleted bioactive composition. The treatment may include administering the bioactive composition to the lungs of a subject with the respiratory condition or disorder, typically by injection, inhalation or spraying, to improve the oxygen-carrying capacity of the lungs, thus treating or aiding in the treatment of a subject with the respiratory condition or disorder.

In some embodiments the enriched cell derived protein fraction can be suspended in a formulation that is compatible with a cannister of a nebulizer inhaler or aspirator. Such formulation can include propellants, or other aerosol producing compounds either as a dry powder inhalation or liquid formulation for nasal and lung delivery, as known in the art.

### Example

In one example, a blood sample is separated to obtain a cellular fraction enriched in alpha-2-macroglobulin (A2M) according to the following procedure.

1. Following centrifugation of a blood sample using the Tropocells® kit and methods, the separated PRP-containing fraction tube is then inverted in-order to suspend the attached cells from the gel-separator.
2. The whole plasma fraction is then filtered to obtain a purified fraction containing non-aggregated cells.
3. The purified fraction containing the platelets, monocytes, and lymphocytes is transferred through a hemoconcentration 15 KDa filter that allows the filtration of proteins that are above 15 kDa to isolate and enrich A2M and other anti-inflammatory proteins while enabling exclusion of water and low molecular weight compounds (under 15 KDa).
4. The purified fraction may be incubated at 37° C. (can be also at Room Temperature) for several minutes to few hours (up to 24 h) with glass beads and/or coated or uncoated plastic beads to obtain a fraction enriched in proteins that are released during incubation. Following the incubation, further concentration of the proteins is employed through use of the hemoconcentrator capable of filtering proteins above 15 kDa.
5. The purified fraction may be suspended with beads or any water adsorbent or surfactant material that may change the osmotic pressure of the cellular fraction to result in protein extraction from the enriched purified fraction.
6. Another option is to enrich the cells that are responsible for secreting anti inflammatory cytokines during the collection of the whole blood to TropoCells® with 1.05-1.09 g/ml gel pre inserted with glass beads or plastic beads inserted above the gel during the incubation that result in the secretion of IL-1Ra and the collection of the enriched fraction via reduction of the cell fraction by centrifugation by applying a 1000 g-4000 g force.
7. Following concentration, the sample is ready to use for application, or cells can be eliminated by further filtration through 0.22 um and/or 0.1 um filter, or by centrifugation for cell precipitation.

### Example

In this Example, the IL-1Ra and A2M found in a PRP sample are quantified and different modes of increasing IL-1Ra and A2M found in the PRP by incubation of a plasma concentration system are compared. The release of IL-Ira and A2M from purified plasma fractions containing leukocytes and platelets was evaluated (1) without incubation; and (2) following conventional incubation at 37° C. for different periods of time: 45 min., 4 h, and 24 h using blood from three donors.

1. In each case (1) and (2), the plasma fraction was obtained using a TropoCells®PRP collection tube and the obtained fractions were each concentrated by removing water using the Hemoconcentrator PROTSMART TM6.
2. Starting cell distribution of white blood cells in whole blood was determined using a Horiba cell counter for each donor using samples stabilized in EDTA-containing test tubes.
3. A 15 ml collection tube was prepared with 1 ml of MNC7 anticoagulant and the tube was filled to the stated 11 ml fill volume with blood from each donor. The filled collection tubes were centrifuged at 7500 RCF (relative centrifugal force) for 10 min, to separate cells from the plasma. This time point immediately after centrifugation is referred to as t=0.
4. Following centrifugation, the tubes were carefully removed from the centrifuge and separated plasma was stored in the freezer at −20° C. until ELISA assessment was obtained.
5. Blood samples from the three donors were collected into two groups of collection tubes (Set A/Set B) until blood flow stopped and each sample reached its stated volume. Set A consisted of pre-coated vacuum tubes with beads. Set B consisted of pre-coated tubes with beads and separator gel. Each group was subjected to identical treatment protocols as described below.
6. Each donor group was divided into three incubation times at 37° C.: t=0, t=4 h, t=24 (overnight). Thus, there were three tubes from each donor for each incubation time for a total of 18 tubes between Set A and Set B.
7. During incubation, each tube was punctured with a vented needle to allow air passage through the butyl rubber stopper. After each incubation, the tubes were aggressively mixed and cell distribution was determined using the Horiba cell counter.
8. Blood from each vacuum tube was transferred into 15 ml tubes. The cells containing plasma were centrifuged at 7500 RCF for 10 min, to eliminate cells from the plasma.
9. All samples were applied on a pre-coated ELISA plate with an IL-Ira, alpha-Macroglobulin (A2M), PDGF-BB, and Human IL-1β/IL-1F2 capture antibodies. The plates were washed according to the manufacturer's instructions and a detection antibody was applied to the plate. The plates were read at a wavelength of 450 nm and an extrapolation standard curve was obtained.
10. FIG. 1 reports a percentage of monocytes in the samples of three donors' blood samples: (1) in whole blood prior to centrifugation; (2) immediately after centrifugation and hemoconcentration in a Medica device (TO); (3) after incubation on beads for four hours followed by hemoconcentration (T4); and (4) after incubation on beads twenty-four hours (T24) followed by hemoconcentration.
11. The IL-1Ra concentration in the incubated plasma samples obtained from Donor 2 are tabulated below, illustrating that incubation increased IL-1Ra concentration in the samples. All samples have a complement of cytokines other than IL-1Ra not present in commercial recombinant products.

TABLE 1

| Incubation Time | IL-1Ra concentration (pg/ml) |
|---|---|
| t = 0 | 1071 |
| t = 4 | 1778 |
| t = 24 | 1849 |

The description of the foregoing preferred embodiments is not to be considered as limiting the invention, which is defined according to the appended claims. The person of ordinary skill in the art, relying on the foregoing disclosure, may practice variants of the embodiments described without departing from the scope of the invention claimed. Where this disclosure refers to “steps” in a process, the steps may be performed in any order, absent a clear indication to the contrary. A feature or dependent claim limitation described in connection with one embodiment or independent claim may be adapted for use with another embodiment or independent claim, without departing from the scope of the invention.

What is claimed is:

1. A method for obtaining blood plasma enriched in bioactive molecules, comprising:
providing a cell suspension collection tube containing a gel separator;
providing a blood sample or cellular suspension in the cell suspension collection tube;
centrifuging the cell suspension collection tube to obtain three fractions in the tube, including: a first fraction containing cells and adapted to be discarded; a second fraction comprising the gel; and a third fraction containing cells and adapted to be recovered; and
extracting a cellular fraction containing blood plasma from the third fraction, wherein said extracting step includes manipulation to induce a release of bioactive molecules in the cellular fraction and/or to reduce or eliminate contaminants in the plasma.

2. The method according to claim 1, comprising incubating cells in the collection tube prior to or after said centrifuging, wherein said incubated cells are adapted to release anti-inflammatory growth factors and cytokines.

3. The method according to any preceding claim, wherein manipulation to induce a release of bioactive molecules is achieved chemically by contacting the cellular fraction with ADP, CaCl2, exogenous or autologous thrombin, or matrix compounds.

4. The method according to any preceding claim, wherein manipulation to induce a release of bioactive molecules is achieved by mechanically fracturing a structural membrane in the cellular suspension.

5. The method according to any preceding claim, wherein manipulation includes subjecting the cellular suspension to sonication, one or more freeze-thaw cycles, or changes in pressure.

6. The method according to any preceding claim, comprising incubating the cellular fraction after centrifugation.

7. The method according to any preceding claim, wherein the blood sample provided in the tube is autologous blood and the cellular fraction extracted from the third fraction is an autologous cellular fraction for treatment.

8. The method according to any preceding claim, comprising filtering the cellular fraction to remove viral contaminants.

9. The method according to any preceding claim, comprising subjecting the cellular fraction to mechanical-physical stress to remove viral contaminants.

10. The method according to any preceding claim, comprising lyophilizing the cellular suspension.

11. A method for treatment of a respiratory condition or disorder, comprising:
obtaining a blood sample;
separating the blood sample in a cell suspension collection tube to obtain a platelet-containing fraction;
activating the platelets in the platelet-containing fraction to enrich the fraction in growth factors and other bioactive molecules;
depleting or removing viruses and viral components from the platelet-containing fraction to form a growth factor-enriched and virus-depleted bioactive composition;
administering the bioactive composition to the lungs of a subject with the respiratory condition or disorder, by injection, inhalation or spraying, to improve the oxygen-carrying capacity of the subject's lungs.
administering the bioactive composition to the lungs of a subject with inflammatory uncontrolled condition by injection to reduce pain and control inflammation.

12. The method according to claim 11, wherein the respiratory condition is caused by Covid-19 infection.

13. The method according any of claims 11-12, further comprising incubating the separated platelet-containing fraction with glass beads and/or coated or uncoated polymer beads.

14. A method for obtaining blood plasma enriched in bioactive molecules and depleted in contaminants, comprising:
providing a blood sample to a cell suspension collection tube containing a gel separator and activators for induction of bioactive molecules;
incubating the collection tube for 30 minutes to 24 hours;
centrifuging the cell suspension collection tube to obtain three fractions in the tube, including: a first fraction containing cells and adapted to be discarded; a second fraction comprising the gel; and a third fraction containing cells, enriched in the bioactive molecules, and adapted to be recovered; and
collecting the third fraction enriched the bioactive molecules.

15. The method according to claim 14, wherein said incubating is performed on said blood sample prior to centrifuging the collection tube.

16. The method according to claim 14, wherein said incubating is performed after centrifuging the collection tube.

17. The method according to any one of claims 14-16, further comprising concentrating the third fraction enriched in the bioactive molecules through filtration with water adsorbent materials.

18. The method according to any one of claims 14-16, further comprising reducing a concentration of contaminants in the third fraction enriched in the bioactive molecules by applying physical-mechanical or chemical-biological stress to the third fraction.

19. The method according to claim 18, wherein the concentration of contaminants is reduced by contacting the third fraction with a solvent or detergent.

20. The method according to claim 18, wherein the concentration of contaminants is reduced by subjecting the third fraction to repeated pressure variations.

21. The method according to claim 18, wherein the concentration of contaminants is reduced by pulsed laser irradiation of the third fraction.

22. The method according to any preceding claim, comprising formulating said third fraction enriched in bioactive molecules as an injection or inhalation formulation

23. The method according to claim 22, wherein the injection or inhalation formulation is adapted to be delivered at the point of care.

24. The method according to claim 23, wherein the injection or inhalation formulation is adapted to be delivered up to six months from cell collection.

25. A method for obtaining blood plasma enriched in bioactive molecules, comprising:
providing a cell suspension collection tube containing a gel separator and incubation beads;
providing a blood sample or cellular suspension in the cell suspension collection tube;
centrifuging the cell suspension collection tube to obtain three fractions in the tube, including: a first fraction containing cells and beads and adapted to be discarded; a second fraction comprising the gel; and a third fraction containing cells and adapted to be recovered; and
extracting a cellular fraction containing blood plasma from the third fraction, wherein said extracting includes increasing a concentration of bioactive molecules in the cellular fraction.

26. The method according to claim 25, wherein said incubation beads are glass beads and/or coated or-coated polymer beads.

27. The method according to claim 25 or 26, wherein increasing a concentration of bioactive molecules in the cellular fraction containing blood plasma includes incubating said cell collection tube.

28. The method according to any one of claims 25-27, further comprising filtering said cellular fraction containing blood plasma to remove water and increase a concentration of bioactive molecules.

29. The method according to any one of claims 25-28, wherein, after said extracting step, said cellular fraction containing blood plasma is increased in a concentration of at least one of alpha-2-macroglobulin, hepatocyte growth factor HGF, transforming growth factor beta TGFβ, platelet derived growth factor PDGF, Interleukin 10 IL₋₁₀, Interleukin 4 IL₋₄, Interleukin 4 receptor antagonist IL₋₄Ra, Interleukin 11 IL₋₁₁, and Interleukin 13 IL₁₃, compared to said third fraction obtained after centrifuging the blood sample or cellular suspension in the cell suspension collection tube.

30. A system for obtaining blood plasma enriched in bioactive molecules, comprising: a cell suspension collection tube containing a gel separation medium and incubation beads, said collection tube adapted to be centrifuged to separate a blood sample and adapted to be incubated for 30 minutes to 24 hours.

31. The system according to claim 30, wherein the gel separation medium has a density between 1.07 g/cm³ to 1.09 g/cm³.

32. The system according to claim 30, wherein said incubation beads are glass beads and/or coated or uncoatedpolymer beads.

33. The system according to claim 30, wherein said collection tube is a vacuum tube.

34. The system according to claim 30, wherein said collection tube further comprises an anticoagulant.
